(19) United States
(12) Patent Application Publication
(10) Pub. No.: US 2020/0069382 A1
(43) Pub. Date: Mar. 5, 2020

(54) SURGICAL INSTRUMENT, ROBOTIC SURGICAL SYSTEM, AND METHOD OF FIXING BEARING- INTEGRATED PULLEY

(71) Applicant: MEDICAROID CORPORATION, Kobe-shi (JP)

(72) Inventors: Yu USUKI, Kobe-shi (JP); Kenji AGO, Kobe-shi (JP)

(73) Assignee: MEDICAROID CORPORATION, Kobe-shi (JP)

(21) Appl. No.: 16/551,650

(22) Filed: Aug. 26, 2019

(30) Foreign Application Priority Data
Aug. 28, 2018 (JP) 2018-159335

(51) Int. Cl. A61B 34/30; A61B 34/37; A61B 34/00

(52) U.S. Cl. CPC A61B 34/30; A61B 2017/00477; A61B 34/71; A61B 34/37

(57) ABSTRACT

A surgical instrument to be detachably connected to a robot arm of the robotic surgical system according to an embodiment may include a base body, a treatment tool, an elongated shaft including a proximal end attached to the base body and a distal end to which the treatment tool is provided, a driven member provided at the base body to be rotatable about a rotational axis with respect to the base body, a bearing-integrated pulley, and an elongated element extending through the shaft to operate the treatment tool. The elongated element is led from the shaft to the driven member through the bearing-integrated pulley and wound around and fixed to the driven member.

### CROSS REFERENCE TO RELATED APPLICATIONS

This application claims priority to Japanese Patent Application No. 2018-159335 filed on Aug. 28, 2018, the entire contents of which are incorporated herein by reference.

### BACKGROUND

The disclosure relates to a surgical instrument, a robotic surgical system, and a method of fixing a bearing-integrated pulley. More specifically, the disclosure may relate to a surgical instrument detachably connected to a robot arm of a robotic surgical system, a robotic surgical system including the surgical instrument, and a method of fixing a bearing-integrated pulley around which a wire is wound to operate a treatment tool provided at a distal end of a shaft of the surgical instruments.

In a related art, a robotic surgical system for assisting surgery has been known. Such a robotic surgical system generally includes a patient-side apparatus with robot arms and a remote control apparatus for remote control of the patient-side apparatus. To the robot arms of the patient-side apparatus, an endoscope and surgical instruments including forceps, for example, are attached. A doctor performs endoscopic surgery for the patient with robot arms of the patient-side apparatus by operating the remote control apparatus while checking patient endoscopic images. Using such a robotic surgical system minimizes the incision in the patient's skin, enabling minimally invasive surgery with the burden on the patient reduced.

In addition, in a related art, surgical instruments detachably connected to the robot arms of such a robotic surgical system have been known (see U.S. Pat. No. 6,394,998, for example). U.S. Pat. No. 6,394,998 discloses surgical equipment (a surgical instrument) that is detachably connected to a robot arm of a robotic surgical system. The surgical equipment includes an end effector, such as forceps, and a spool around which a cable used to operate the end effector is wound. The end effector is provided at a distal end of an elongated shaft and the spool is provided at a proximal end side of the shaft. Further, the shaft is formed being hollow so that the cable can be inserted. The cable is led from the shaft via a guide pulley provided near the spool to the spool and wound around and fixed to the spool. Accordingly, the surgical equipment is configured so that the end effector provided at the distal end of the shaft is operated to move by rotating the spool to move the cable through the guide pulley.

### SUMMARY

If a folding angle of the cable at the guide pulley (an angle between before the direction of the cable is changed at the guide pulley and after the direction of the cable is changed at the guide pulley) is large, tension of the cable applied to the guide pulley becomes large and thus the pulley (guide pulley) may not smoothly rotate according to the movement of the cable. In view of this, it is not preferable that the driven member around which the cable is wound and fixed is provided at a position where the folding angle of the cable at the guide pulley becomes large. Therefore, the arrangement of the driven member may be restricted, and this may make it difficult to downsize the surgical equipment due to the restriction of the arrangement of the driven member.

An object of an embodiment of the disclosure may be to provide a surgical instrument that can improve flexibility in arrangement of the driven member and downsize the surgical instrument, a robotic surgical system including the surgical instrument, and a method of fixing a bearing-integrated pulley around which a wire is wound to operate the treatment tool provided at a distal end of a shaft of the surgical instrument.

A surgical instrument to be detachably connected to a robot arm of a robotic surgical system according to a first aspect of the disclosure may include: a base body; a treatment tool; a shaft whose proximal end is attached to the base body and whose distal end is provided with the treatment tool; a driven member provided in the base body to be rotatable about a rotational axis with respect to the base body; a bearing-integrated pulley; and an elongated element extending through the shaft to operate the treatment tool. The elongated element is led from the shaft to the driven member through the bearing-integrated pulley and wound around and fixed to the driven member.

A robotic surgical system according to a second aspect of the disclosure may include a robot arm and a surgical instrument that is detachably connected to the robot arm. The surgical instrument may include: a base body; a treatment tool; a shaft whose proximal end is attached to the base body and whose distal end is provided with the treatment tool; a driven member provided at the base body to be rotatable about a rotational axis with respect to the base body; a bearing-integrated pulley, and an elongated element extending through the shaft to operate the treatment tool. The elongated element is led from the shaft to the driven member through the bearing-integrated pulley and wound around and fixed to the driven member.

A method according to a third aspect of the disclosure may be a method to fix a bearing-integrated pulley around which a wire for operating the treatment tool is wound. The treatment tool may be provided at a distal end of the shaft of the surgical instruments, which is detachably connected to the robot arm. The method according to the third aspect may include: retaining a pulley shaft part of the bearing-integrated pulley by inserting the pulley shaft part of the bearing-integrated pulley into a retaining groove formed on a pulley retainer; and pressing the pulley shaft part onto a side face of the retaining groove using tension of the wire.

### BRIEF DESCRIPTION OF DRAWINGS

FIG. 1 is a diagram illustrating an overview of a robotic surgical system according to an embodiment.

FIG. 2 is a block diagram illustrating a view of a control-related configuration of the robotic surgical system according to an embodiment.

FIG. 3 is a diagram illustrating a perspective view of a state where a surgical instrument is attached to the robot arm through an adaptor according to an embodiment.

FIG. 4 is a diagram illustrating a perspective view of a state where the surgical instrument and the adaptor are detached from the robot arm according to an embodiment.

FIG. 5 is a diagram illustrating a perspective view of the adaptor and surgical instrument according to an embodiment as seen in Z2 direction.

FIG. 6 is a diagram illustrating a perspective view of a state where a cover part is detached from a base body of the surgical instrument according to an embodiment.

FIG. 7 is a diagram illustrating a view of the state where the cover part is detached from the base body of the surgical instrument according to an embodiment, seen in Z1 direction.

FIG. 8 is a diagram illustrating a perspective view of a treatment tool of the surgical instrument according to an embodiment.

FIG. 9A is a diagram illustrating a perspective view of a first bearing-integrated pulley of the surgical instrument according to an embodiment.

FIG. 9B is a diagram illustrating an exploded perspective view of the first bearing-integrated pulley of the surgical instrument according to an embodiment.

FIG. 10A is a diagram illustrating a perspective view of a second bearing-integrated pulley of the surgical instrument according to an embodiment.

FIG. 10B is a diagram illustrating an exploded perspective view of the second bearing-integrated pulley of the surgical instrument according to an embodiment.

FIG. 11A is a diagram illustrating a perspective view of a third bearing-integrated pulley of the surgical instrument according to an embodiment.

FIG. 11B is a diagram illustrating an exploded perspective view of the third bearing-integrated pulley of the surgical instrument according to an embodiment.

FIG. 12 is a diagram illustrating a view of the first bearing-integrated pulley, second bearing-integrated pulley, third bearing-integrated pulley, and a pulley retainer of the surgical instrument according to an embodiment, seen in the Z1 direction.

FIG. 13 is a diagram illustrating a schematic cross-sectional view along the line 500-500 in FIG. 12.

FIG. 14 is a diagram illustrating a schematic cross-sectional view along the line 600-600 in FIG. 12.

### DETAILED DESCRIPTION

Descriptions are provided hereinbelow for one or more embodiments based on the drawings. In the respective drawings referenced herein, the same constituents are designated by the same reference numerals and duplicate explanation concerning the same constituents is omitted. All of the drawings are provided to illustrate the respective examples only.

### (Configuration of Robotic Surgical System)

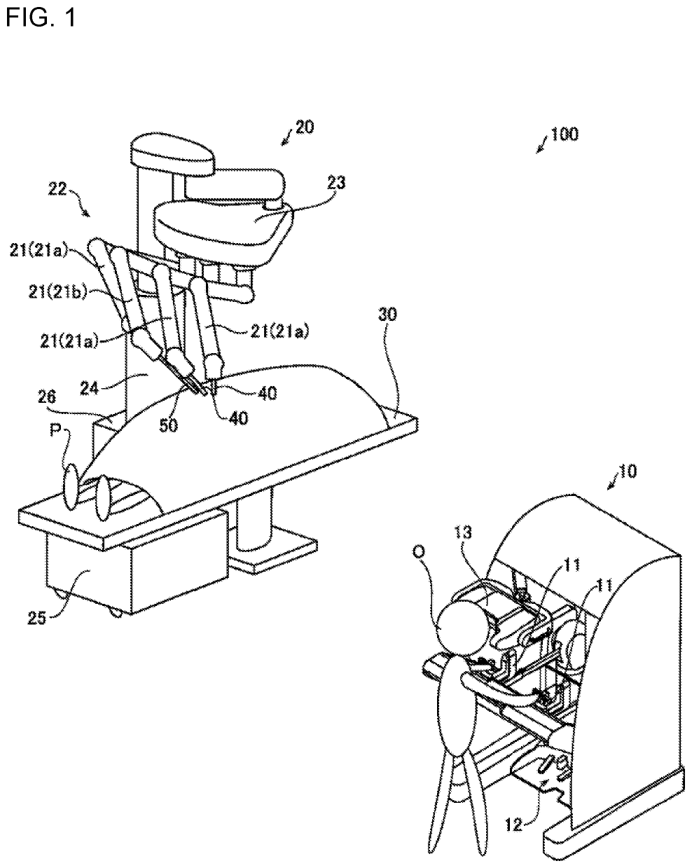

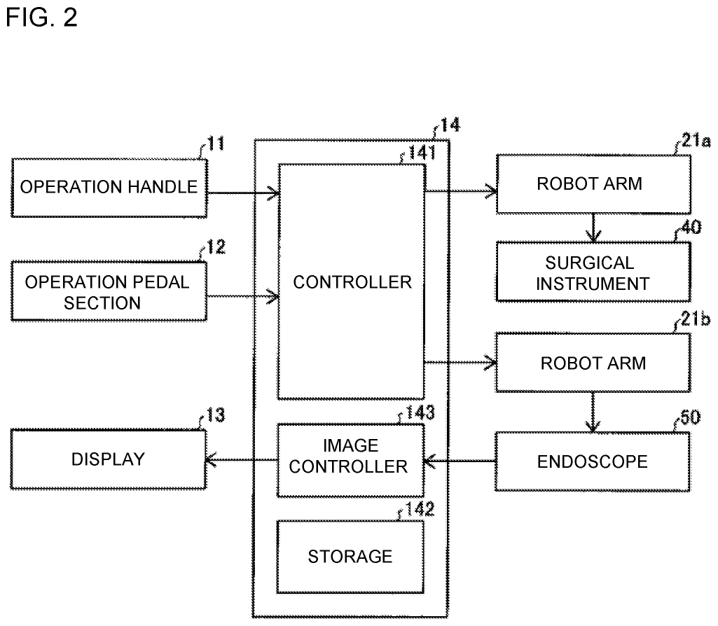

A configuration of a robotic surgical system 100 according to an embodiment is described with reference to FIGS. 1 and 2.

As illustrated in FIG. 1, the robotic surgical system 100 includes a remote control apparatus 10 and a patient-side apparatus 20. The remote control apparatus 10 is provided to remotely control medical equipment provided for the patient-side apparatus 20. When an operator O, as a surgeon, inputs an action mode instruction to be executed by the patient-side apparatus 20, to the remote control apparatus 10, the remote control apparatus 10 transmits the action mode instruction to the patient-side apparatus 20 through a controller 26. In response to the action mode instruction transmitted from the remote control apparatus 10, the patient-side apparatus 20 operates medical equipment such as surgical instruments 40, an endoscope 50, and the like, attached to robot arms 21. This allows for minimally invasive surgery.

The patient-side apparatus 20 constitutes an interface to perform a surgery for a patient P. The patient-side apparatus 20 is positioned beside an operation table 30 on which the patient P is laid. The patient-side apparatus 20 includes plural robot arms 21. One of the robot arms 21 (21b) holds the endoscope 50 while the other robot arms 21 (21a) hold the surgical instruments 40. The robot arms 21 are commonly supported by a platform 23. Each of the plural robot arms 21 includes plural joints. Each joint includes a driver provided with a servo-motor and a position detector such as an encoder. The robot arms 21 are configured so that the medical equipment attached to each robot arm 21 is controlled by a driving signal given through the controller 26 and performs a desired movement.

The platform 23 is supported by a positioner 22 placed on the floor of an operation room. The positioner 22 includes a column 24 and a base 25. The column 24 includes an elevating shaft adjustable in the vertical direction. The base 25 includes wheels and is movable on the floor surface.

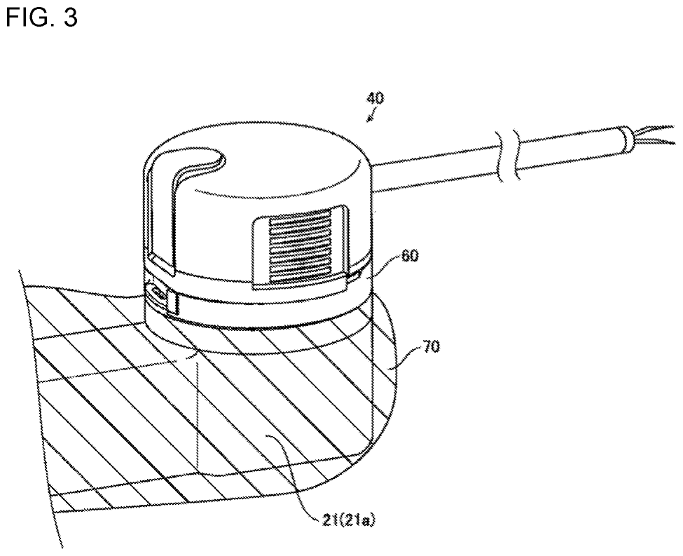

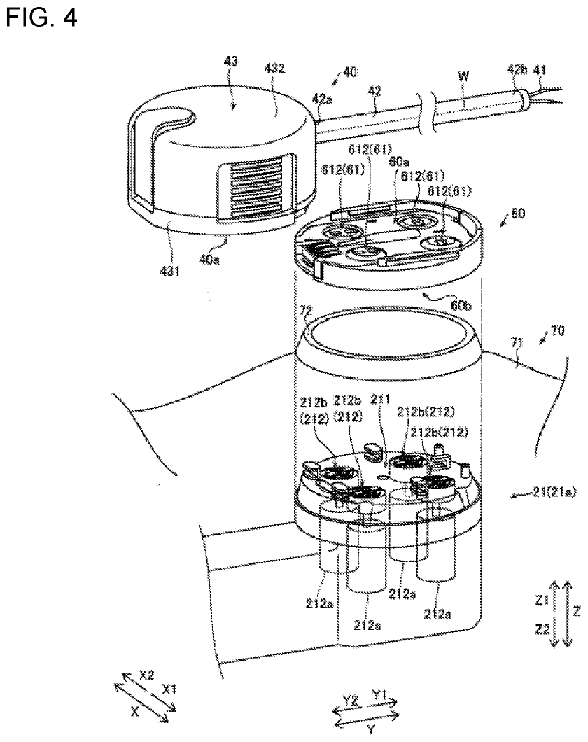

The surgical instruments 40 as the medical equipment is detachably attached to the distal ends of the robot arms 21a. Each surgical instrument 40 includes: a base body 43 (see FIG. 4), which is attached to the robot arm 21a; an elongated shaft 42 (see FIG. 4); and a treatment tool 41 (an end effector) (see FIG. 4), which is provided at the tip of the shaft 42. The treatment tool 41 is grasping forceps, scissors, a hook, a high-frequency knife, a snare wire, a clamp, or a stapler, for example. The end effector 41 is not limited to those and can be various types of treatment tools. In surgeries using the patient-side apparatus 20, the robot arms 21a introduce the surgical instruments 40 into the body of the patient P through a cannula (trocar) placed on the body surface of the patient P. The treatment tools 41 of the surgical instruments 40 are then located near the surgery site.

To the distal end of the robot arm 21b, the endoscope 50 as the medical equipment is detachably attached. The endoscope 50 captures an image within the body cavity of the patient P. The captured image is outputted to the remote control apparatus 10. The endoscope 50 is a 3D endoscope capable of capturing a three-dimensional image or a 2D endoscope. In surgeries using the patient-side apparatus 20, the robot arm 21b introduces the endoscope 50 into the body of the patient P through a trocar placed on the body surface of the patient P. The endoscope 50 is then located near the surgery site.

The remote control apparatus 10 constitutes the interface with the operator O. The remote control apparatus 10 is an apparatus that allows the operator O to operate medical equipment attached to the robot arms 21. Specifically, the remote control apparatus 10 is configured to transmit action mode instructions which are inputted by the operator O and are to be executed by the surgical instruments 40 and endoscope 50, to the patient-side apparatus 20 through the controller 26. The remote control apparatus 10 is installed beside the operation table 30 so that the operator O can see the condition of the patient P very well while operating the remote control apparatus 10, for example. The remote control apparatus 10 may be configured to transmit action mode instructions wirelessly and installed in a room different from the operation room where the operation table 30 is installed.

The action modes to be executed by the surgical instruments 40 include modes of actions to be taken by each surgical instrument 40 (a series of positions and postures) and actions to be executed by the function of each surgical instrument 40. When the surgical instrument 40 is a pair of grasping forceps, for example, the action modes to be executed by the surgical instrument 40 include roll and pitch positions of the wrist of the treatment tool 41 and actions to open and close the jaws. When the surgical instrument 40 is a high-frequency knife, the action modes to be executed by the surgical instrument 40 include vibration of the high-frequency knife, specifically, supply of current to the high-frequency knife. When the surgical instrument 40 is a snare wire, the action modes to be executed by the surgical instrument 40 include a capturing action and an action to release the captured object and include an action to supply current to a bipolar or monopolar instrument to burn off the surgery site.

The action modes to be executed by the endoscope 50 include the position and posture of the tip of the endoscope 50 and setting of the zoom magnification, for example.

As illustrated in FIGS. 1 and 2, the remote control apparatus 10 includes operation handles 11, an operation pedal section 12, a display section 13, and a control apparatus 14.

The operation handles 11 are provided in order to remotely operate medical equipment attached to the robot arms 21. Specifically, the operation handles 11 accept operations by the operator O for operating medical equipment (the surgical instruments 40 and endoscope 50). The operation handles 11 include two operation handles 11 arranged side by side in the horizontal direction. One of the two operation handles 11 is operated by the right hand of the operator O while the other operation handle 11 is operated by the left hand of the operator O.

The operation handles 11 extend from the rear side of the remote control apparatus 10 toward the front side. The operation handles 11 are configured to move in a predetermined three-dimensional operation region. Specifically, the operation handles 11 are configured so as to move up and down, right and left, and forward and rearward.

The remote control apparatus 10 and patient-side apparatus 20 constitute a master-slave system in terms of controlling movement of the robot arms 21a and robot arm 21b. The operation handles 11 constitute an operating section or an operating part on the master side in the master-slave system, and the robot arms 21a and 21b holding medical equipment constitute an operating section or an operation part on the slave side. When the operator O operates the operation handles 11, the movement of one of the robot arms 21a or 21b is controlled so that the tip (the treatment tool 41 of the surgical instrument 40) of the robot arm 21a or the tip (the endoscope 50) of the robot arm 21b moves following the movement of the operation handles 11.

The patient-side apparatus 20 controls the movement of the robot arms 21a in accordance with the set motion scaling ratio. When the motion scaling ratio is set to ½, for example, the treatment tool 41 of the surgical instruments 40 move ½ of the movement distance of the operation handles 11. This allows for precise fine surgery.

The operation pedal section 12 or an operation pedal unit includes plural pedals to execute medical equipment-related functions. The plural pedals include a coagulation pedal, a cutting pedal, a camera pedal, and a clutch pedal. The plural pedals are operated by a foot of the operator O.

The coagulation pedal enables the surgical instrument 40 to coagulate a surgery site. Specifically, when the coagulation pedal is operated, voltage for coagulation is applied to the surgical instrument 40 to coagulate a surgery site. The cutting pedal enables the surgical instrument 40 to cut a surgery site. Specifically, the cutting pedal is operated to apply voltage for cutting to the surgical instrument 40 and cut a surgery site.

The camera pedal is used to control the position and orientation of the endoscope 50 that captures images within the body cavity. Specifically, the camera pedal enables operation of the endoscope 50 by the operation handles 11. The position and orientation of the endoscope 50 are controllable by the operation handles 11 while the camera pedal is being pressed. The endoscope 50 is controlled by using both of the right and left operation handles 11, for example. Specifically, when the operator O rotates the right and left operation handles 11 about the middle point between the right and left operation handles 11, the endoscope 50 is rotated. When the operator O presses the right and left operation handles 11 together, the endoscope 50 goes forward into the body cavity. When the operator O pulls the right and left operation handles 11 together, the endoscope 50 goes back. When the operator O moves the right and left operation handles 11 together up, down, right, or left, the endoscope 50 moves up, down, right, or left, respectively.

The clutch pedal is used to temporarily disconnect operation-related connection between the operation handles 11 and the robot arms 21 to stop movement of the surgical instruments 40. Specifically, when the clutch pedal is being pressed, the robot arms 21 of the patient-side apparatus 20 do not work even if the operation handles 11 are operated. For example, when the operation handles 11 are operated and moved to the edge of the range of movement, the operator O operates the clutch pedal to temporarily disconnect the operation-related connection and then returns the operation handles 11 to the center of the range of movement. When the operator O stops operating the clutch pedal, the operation handles 11 are again connected to the robot arms 21. The operator O restarts the operation for the operation handles 11 around the center thereof.

The display section 13 or a display unit is configured to display images captured by the endoscope 50. The display section 13 includes a scope type display section or a non-scope type display section. The scope type display section is a display section that the operator O looks into. The non-scope type display section is a display section like an open-type display section that includes a flat screen and the operator O is able to see without looking into, such as normal displays for personal computers.

When the scope type display section is attached, the scope type display section displays 3D images captured by the endoscope 50 attached to the robot arm 21b of the patient-side apparatus 20. When the non-scope type display section is attached, the non-scope type display section also displays 3D images captured by the endoscope 50 provided for the patient-side apparatus 20. The non-scope type display section may display 2D images captured by the endoscope 50 provided for the patient-side apparatus 20.

As illustrated in FIG. 2, the control apparatus 14 includes a controller 141, a storage 142, and an image controller 143, for example. The controller 141 includes a calculator such as a CPU. The storage 142 includes a memory, such as a ROM and a RAM. The control apparatus 14 may be composed of a single controller performing centralized control or may be composed of plural controllers that perform decentralized control in cooperation with each other. The controller 141 determines whether an action mode instruction inputted by the operation handles 11 is to be executed by the robot arms 21a or to be executed by the endoscope 50, depending on the state of the operation pedal section 12. When determining that the action mode instruction inputted by the operation handles 11 is to be executed by any one of the surgical instruments 40, the controller 141 transmits the action mode instruction to the corresponding robot arm 21a. The robot arm 21a is thereby driven for controlling movement of the surgical instrument 40 attached to the robot arm 21a.

When determining that the action mode instruction inputted by the operation handles 11 is to be executed by the endoscope 50, the controller 141 transmits the action mode instruction to the robot arm 21b. The robot arm 21b is thereby driven for control of movement of the endoscope 50 attached to the robot arm 21b.

The storage 142 stores control programs corresponding to the types of the surgical instrument 40, for example. The controller 141 reads the stored control programs according to the types of the attached surgical instruments 40. The action mode instructions from the operation handles 11 and/or the operation pedal section 12 of the remote control apparatus 10 thereby cause the respective surgical instruments 40 to perform proper movements.

The image controller 143 transmits images acquired by the endoscope 50 to the display section 13. The image controller 143 performs processing and alternations for the images when needed.

### (Configuration of Surgical Instrument, Adaptor, Drape, and Robot Arm)

With reference to FIGS. 3 to 14, the configurations of the surgical instrument 40, adaptor 60, drape 70, and robot arm 21 according to an embodiment are described.

### <Attachment Condition>

As illustrated in FIGS. 3 to 5, the surgical instrument 40 is detachably connected to the robot arm 21 through the adaptor 60. The adaptor 60 is a drape adaptor configured to sandwich a sterile drape 70 to cover the robot arm 21, in conjunction with the robot arm 21. The adaptor 60 is attached to an attachment surface 40a of a base body 43, on the Z2 side of the surgical instrument 40. The surgical instrument 40 is attached to an attachment surface 60a of the adaptor 60 on the Z1 side. The robot arm 21 is attached to an attachment surface 60b of the adaptor 60 on the Z2 side. The adaptor 60 is attached to an attachment surface 211 of the robot arm 21 on the Z1 side.

The robot arm 21 is used in a clean area and is covered with the drape 70. In operation rooms, clean technique is used in order to prevent surgical incision sites and medical equipment from being contaminated by pathogen, foreign matters, or the like. The clean technique defines a clean area and a contaminated area, which is other than the clean area. The surgery sites are located in the clean area. Members of the surgical team, including the operator O, make sure that only sterile objects are placed in the clean area during surgery and perform sterilization for an object which is to be moved to the clean area from the contaminated area. Similarly, when the members of the surgical team including the operator O place their hands in the contaminated area, the members sterilize their hands before directly touching objects located in the clean area. Instruments used in the clean area are sterilized or are covered with sterile drape 70.

The drape 70 includes a body section 71 and an attachment section 72. The body section 71 covers the robot arm 21. The attachment section 72 is sandwiched between the robot arm 21 and adaptor 60. The body section 71 is made of a flexible film member. The flexible film member is made of a resin material, such as thermoplastic polyurethane and polyethylene. The body section 71 includes an opening so that the robot arm 21 is engaged with the adaptor 60. In the opening of the body section 71, the attachment section 72 is provided so as to close the opening. The attachment section 72 is made of a resin mold member. The resin mold member is made of a resin member such as polyethylene terephthalate. The attachment section 72 is harder (less flexible) than the body section 71. The attachment section 72 includes an opening so that the robot arm 21 is engaged with the adaptor 60. The opening of the attachment section 72 may be provided corresponding to the section where the robot arm 21 is engaged with the adaptor 60. The opening of the attachment section 72 may include plural openings corresponding to respective plural sections at which the robot arm 21 is engaged with the adaptor 60.

### <Configuration Related to Driving of Treatment Tool>

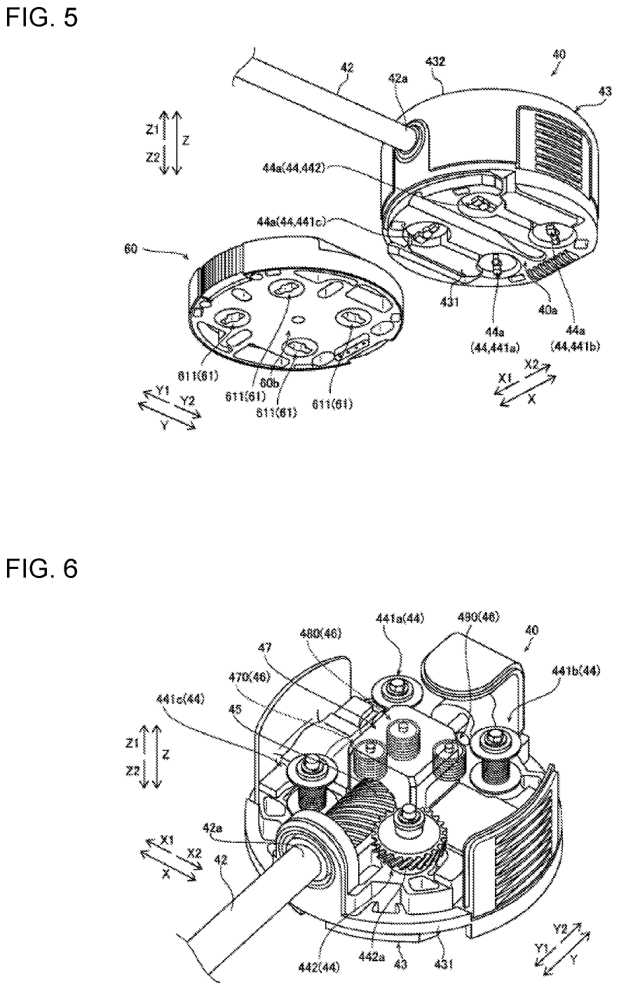

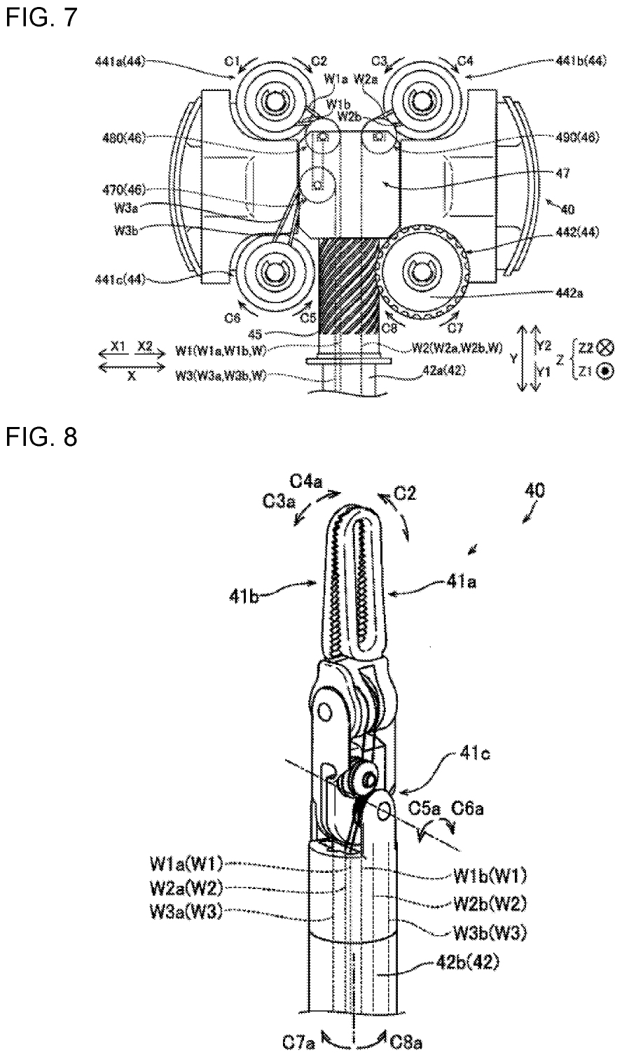

As illustrated in FIGS. 5 to 7, the surgical instruments 40 includes plural (four) driven members 44. Each of the driven members 44 is rotatable respective to the base body 43 about a rotary shaft extending in the Z direction. The base body 43 includes a resin base part 431 and a resin cover part 432. The cover part 432 is provided to cover the base part 431. The plural driven members 44 include plural (three) driven members 441a to 441c and a driven member 442. Wires W (see FIG. 7) for operating (driving) the treatment tool 41 are wound around and fixed to the driven members 441a to 441c. The driven member 442 includes a gear portion 442a for operating (driving) the treatment tool 41. Note that each of the wires W may be an example of an elongated element. The driven member 441a may be an example of a second driven member. The driven member 441b may be an example of a third driven member. The driven member 441c may be an example of a first driven member.

A midway of each of the wires W is folded and disposed on a distal end side (in the Y1 side) of the shaft 42 and is fixed to a movable part of the treatment tool 41. Each wire W extends through the hollow shaft 42 in the Y direction and two ends of the wire W are drawn from the hollow shaft 42 to the proximal end side (in the Y2 side) and wound around and fixed to the corresponding one of the driven members 441a,441b, and 441c. A proximal end 42a of the shaft 42 is attached to the base body 43 and, a distal end 42b of the shaft 42 is provided with the treatment tool 41. Specifically, the midway of each wire W, which is located on the distal side, is folded to form in a substantially U-shape. An attachment (not shown) is fixed to the midway folded part of each wire W and is fixed to the treatment tool 41. Both end portions or both sides of each wire W, which are disposed on the proximal side (the Y2 side) of the shaft 42, are respectively drawn from the shaft 42 to corresponding one of the driven members 441a 441b, and 441c and wound around and fixed to the corresponding one of the driven members 441a 441b, and 441c to operate the treatment tool 41. According to rotation of the driven members 441a to 441c, the wires W are driven to move and, the treatment tool 41 is operated according to the movements of the wires W. Each of the wires W is made of a metal material such as stainless steel, tungsten, or the like. The wires W comprises the number of wires corresponding to respective types of the treatment tool 41. For example, the wires W includes wires W1 (W1a, W1b) and W2 (W2a, W2b) for operating to open and close a pair of jaws 41a and 41b (see FIG. 8) of the treatment tool 41 and a wire W3 (W3a, W3b) for operating to rotate a wrist portion 41c (see FIG. 8) of the treatment tool 41. The wire W1 may be an example of a second wire. The wire W2 may be an example of a third wire. The wire W3 may be an example of a first wire.

As illustrated in FIGS. 7 and 8, both end portions W1a and W1b of the wire W1 (or both sides W1a and W1b of the wire W1) are led from the shaft 42 to the driven member 441a and wound around and fixed to the driven member 441a. In this case, when the driven member 441a rotates about the rotary shaft thereof, the rotation of the driven member 441 operates the jaw 41a of the pair of jaws 41a and 41b of the treatment tool 41 with the wire W1. Specifically, when the driven member 441a rotates in the C1 direction (see FIG. 7), the jaw 41a is driven to move in the C1a direction (see FIG. 8) which is a direction that the jaw 41a opens. When the driven member 441a rotates in the C2 direction (see FIG. 7) which is opposite to the C1 direction, this rotation drives the jaw 41a to move in the C2a direction (see FIG. 8) that the jaw 41a closes.

The wires W2a and W2b (end portions W2a and W2b of the wire W2 or both sides W2a and W2b of the wire W2) are led from the shaft 42 to the driven member 441b and wound around and fixed to the driven member 441b, for example. In this case, when the driven member 441b rotates about the rotary shaft thereof, the rotation of the driven member 441b operates, with the wire W2, the jaw 41b, which is one of the pair of jaws 41a and 41b of the treatment tool 41. Specifically, when the driven member 441b rotates in the C3 direction (see FIG. 7), the jaw 41b is driven to move in the C3a (see FIG. 8) that the jaw 41b opens. When the driven member 441b rotates in the C4 direction (see FIG. 7) which is opposite to the C3 direction, the jaw 41b is driven to move in the C4a direction (see FIG. 8) that the jaw 41b closes.

The wires W3a and W3b (both end portions W3a and W3b of the wire W3 or both sides W3a and W3b of the wire W3) are drawn from the shaft 42 to the driven member 441c and wound around and fixed to the driven member 441c, for example. In this case, when the driven member 441c rotates about the rotary shaft thereof, the rotation of the driven member 441c operates the wrist portion 41c of the treatment tool 41 with the wire W3. Specifically, when the driven member 441c rotates in the C5 direction (see FIG. 7), the wrist portion 41c is driven to move in the C5a direction (see FIG. 8). When the driven member 441c rotates in the C6 direction (see FIG. 7), which is opposite to the C5 direction, the wrist portion 41c is driven to move in the C6a direction (see FIG. 8), which is opposite to the C5a direction.

When the driven member 442 rotates about the rotary shaft thereof with the gear portion 442a of the driven member 442 being engaged with a gear portion 45 connected to the proximal end 42a of the shaft 42, the shaft 42 is driven to operate the treatment tool 41. Specifically, when the driven member 442 rotates in the C7 direction (see FIG. 7), the shaft 42 is driven to rotate in the C7a direction (see FIG. 8) and thus the treatment tool 41 is driven to rotate in the C7a. When the driven member 442 rotates in the C8 direction (see FIG. 7), the shaft 42 is driven to rotate in the C8a direction (see FIG. 8), which is opposite to the C7a direction, and thus the treatment tool 41 is driven to rotate in the C8a direction.

As illustrated in FIGS. 4 and 5, the robot arm 21 generates driving force for driving the treatment tool 41 of the surgical instruments 40 and transmits the driving force to the surgical instruments 40 through the adaptor 60. Specifically, the robot arm 21 includes plural (four) drive members 212 provided corresponding to the plural (four) driven members 44 of the surgical instruments 40. Each drive member 212 includes an actuator 212a and an engagement protrusion 212b. The actuator 212a includes a motor to drive the drive member 212. The engagement protrusion 212b is rotated by the actuator 212a about the rotary shaft thereof extending in the Z direction. Each engagement protrusion 212b protrudes from a surface of the drive member 212 on the Z1 side toward the adaptor 60 side (in the Z1 side). Engagement protrusions 212b are provided corresponding to engagement recesses 611 of later described drive transmission members 61 of the adaptor 60.

The adaptor 60 includes plural (four) drive transmission members 61 provided corresponding to the plural (four) driven members 44 of the surgical instruments 40. The drive transmission members 61 are rotatable about a rotary shaft thereof extending in the Z direction. Each drive transmission member 61 includes an engagement recess 611 (see FIG. 5). The engagement recess 611 engages with the engagement protrusion 212b of the robot arms 21. The engagement recess 611 is provided at a surface of the drive transmission member 61 on the Z2 side (the robot arm 21 side) and is recessed from the Z2 side surface of the drive transmission member 61, in the Z1 direction, opposite to the robot arm 21 side. Each drive transmission member 61 also includes an engagement recess 612 (see FIG. 4). Each engagement recess 612 engages with a later described engagement protrusion 44a of the driven member 44 of the surgical instruments 40. The engagement recess 612 is provided at a surface of the drive transmission member 61 on the Z1 side (the surgical instruments 40 side) and is recessed from the Z1 side surface of the drive transmission member 61 toward a direction (in the Z2 direction) opposite to the surgical instruments 40 side.

Each driven member 44 of the surgical instrument 40 includes an engagement protrusion 44a. The engagement protrusions 44a engages with the engagement recesses 612 of the drive transmission member 61. Each engagement protrusion 44a protrudes from the Z2 side surface of the driven member 44 toward the adaptor 60 side (in the Z2 direction). In the state where the drive members 212 of the robot arm 21 are engaged with the drive transmission members 61 of the adaptor 60 and the drive transmission members 61 of the adaptor 60 are engaged with the driven members 44 of the surgical instruments 40, the drive transmission members 61 of the adaptor 60 can transmit driving force by the actuators 212a of the robot arm 21 to the driven members 44 of the surgical instrument 40.

The engagement recesses 612 of the drive transmission members 61 on the Y1 side are different in shape from the engagement recesses 612 of the drive transmission members 61 on the Y2 side. Regarding the engagement protrusions 44a which engage with the engagement recesses 612, the engagement recesses 612 of the driven members 44 on the Y1 side are different in shape from the engagement recesses 612 of driven members 44 on the Y2 side. This configuration prevents the engagement protrusions 44a of the driven members 44 on the Y1 side and the engagement recesses 612 of the drive transmission members 61 on the Y2 side from being engaged and stacked, when the base body 43 of the surgical instrument 40 is attached to the adaptor 60 as being slid in the Y1 direction. As a result, the surgical instrument 40 can be easily attached to the adaptor 60.

### <Configuration of Bearing-Integrated Pulleys of Surgical Instrument>

As illustrated in FIGS. 6 and 7, the surgical instrument 40 includes plural (three) bearing-integrated pulleys 46. Each of the plural (three) bearing-integrated pulleys 46 is rotatable with respect to the base body 43 about a rotational axis thereof extending in the Z direction. The wires W, extending through the shaft 42 in the Y direction and led out from the proximal end 42a of the shaft 42 in the Y2 direction, are wound around the plural bearing-integrated pulleys 46. The plural bearing-integrated pulleys 46 guide the wires W led from the shaft 42 to the driven members 441a to 441c.

According to an embodiment, the wires W are led from the shaft 42 via the bearing-integrated pulleys 46 to the driven members 441a to 441c and wounded around and fixed to the driven members 441a to 441c. With this configuration, even when a folding angle of the wires W at the bearing-integrated pulleys 46 is large and tension applied to the bearing-integrated pulleys 46 from the wires W is also large, the pulleys (bearing-integrated pulleys 46) around which the wires W are wound can be smoothly rotated along with movements of the wires W, by the function of the integrated bearing. As a result, the driven members 441a to 441c around which the wires W are wound and fixed can be provided even in a position where the folding angle of the wires W at the bearing-integrated pulley 46 is large. This makes the surgical instrument 40 compact since flexibility in arrangement of the driven members 441a to 441c improves.

According to an embodiment, the plural (three) bearing-integrated pulleys 46 include a bearing-integrated pulley 470, a bearing-integrated pulley 480, and a bearing-integrated pulley 490 corresponding to the respective plural (three) driven members 441a to 441c around which the wires W are wound and fixed. With this configuration, the respective bearing-integrated pulleys 46 (470, 480, and 490) can be made smaller in size compared to a case where only one or two bearing-integrated pulleys 46 are provided. This is effective to prevent an increase in size in the Z direction of the surgical instrument 40 in a case of using the bearing-integrated pulleys 46 with a larger thickness (the length in the Z direction) of a later described pulley part (bearing-integrated pulley parts 472, 473, 482, 483, 492, and 493) than that of a normal pulley. The bearing-integrated pulleys 46 are provided at positions corresponding to the plural driven members 441a to 441c, such that the number of the bearing-integrated pulleys 46 corresponds to the number of the driven members 441a to 441c. Here, the bearing-integrated pulleys 470, 480 and 490 may be examples of a first bearing-integrated pulley, a second bearing-integrated pulley, and a third bearing-integrated pulley, respectively.

The bearing-integrated pulley 470 is provided corresponding to the driven member 441c. The bearing-integrated pulley 470 is positioned in an opposite side of the shaft 42 (in the Y2 side) with respect to the driven member 441c in the direction that the shaft 42 extends (the Y direction). Around the bearing-integrated pulley 470, the wire W3, which is drawn in the Y2 direction from the proximal end 42a of the shaft 42, is wound as being folded to a direction (in the Y1 side, the shaft 42 side) opposite from the Y2 side.

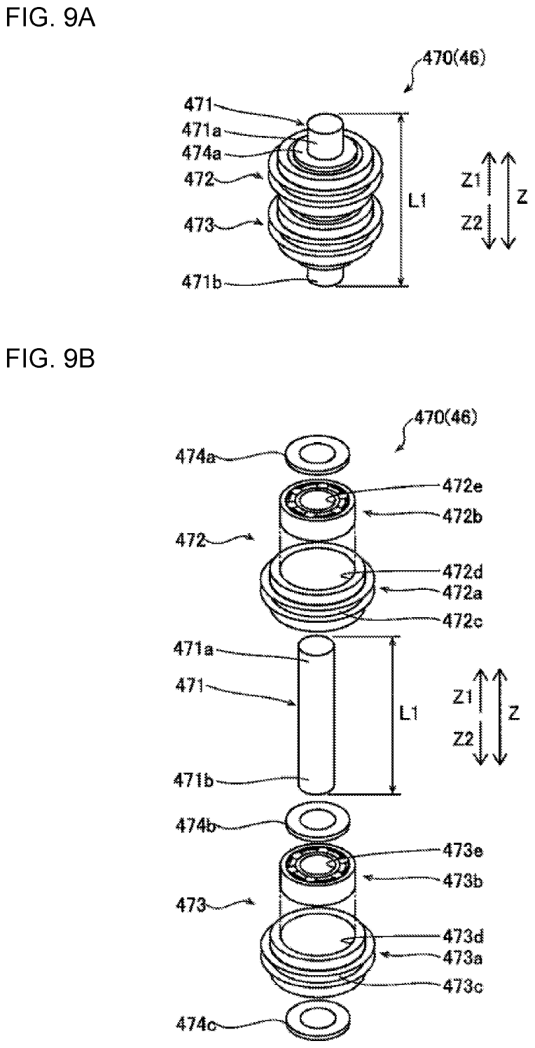

As illustrated in FIGS. 9A and 9B, the bearing-integrated pulley 470 includes a pulley shaft part 471, plural (two) bearing-integrated pulley parts 472 and 473, and plural (three) washers 474a to 474c. The pulley shaft part 471 extends in the Z direction. The pulley shaft part 471 has a length L1 in the Z direction (the axial direction of the shaft). The length L1 is shorter than a length L2 (L3) of a later described pulley shaft part 481 (491) of the bearing-integrated pulley 480 (490) in the Z direction. The bearing-integrated pulley part 472 is provided on (inserted therethrough) the pulley shaft part 471. The bearing-integrated pulley part 473 is also provided on (inserted therethrough) the pulley shaft part 471 such that the bearing-integrated pulley parts 472 and 473 are provided side by side in the axial direction (the Z direction). The bearing-integrated pulley parts 472 and 473 are assembled to the pulley shaft part 471 in that order from the Z1 side toward the Z2 side. The first wire W3a and second wire W3b of the wire W3 are led from the shaft 42 through the bearing-integrated pulley parts 472 and 473 to the driven member 441c and wound around and fixed to the driven member 441c. With this configuration, the bearing-integrated pulley parts 472 and 473 around which the wires W3a and W3b are wound can be smoothly rotated along with movements of the wires W3a and W3b by the function of the integrated bearing, even when tension of the wires W3a and W3b applied to the bearing-integrated pulley 470 is large.

Specifically, the first wire W3a (see FIGS. 7 and 13), which is one side part of the wire W3, is wound around the bearing-integrated pulley part 472 to be folded in a substantially U-shape. The second wire W3b (see FIGS. 7 and 13), which is the other side part of the wire W3, is wound around the bearing-integrated pulley part 473 to be folded in a substantially U-shape. The bearing-integrated pulley parts 472 and 473 are composed of same components (same members).

The bearing-integrated pulley part 472 (473) includes a pulley part 472a (473a) and a bearing part 472b (473b). The pulley part 472a includes a pulley groove 472c (473c) on its outer circumference. Around the pulley groove 472c (473c), the first wire W3a (second wire W3b) of the wire W3 is wound. The pulley part 472a (473a) includes a through-hole 472d (473d) penetrating in the Z direction and forming in an inner circumference of the pully part. The bearing part 472b (473b) is inserted in and fitted to the through-hole 472d (473d). The bearing part 472b (473b) is for example a ball bearing and its outer circumference is fitted to the through-hole 472d (473d) of the pulley part 472a (473a). The bearing part 472b (473b) holds the pulley part 472a (473a) rotatably about the rotary shaft extending in the Z direction. The bearing part 472b (473b) includes a through-hole 472e (473e) which penetrates in the Z direction. The pulley shaft part 471 is inserted in and fitted to the through-hole 472e (473e).

The washers 474a to 474c are respectively provided on the Z1 side of the bearing-integrated pulley part 472, between the bearing-integrated pulley part 472 and the bearing-integrated pulley part 473, and on the Z2 side of the bearing-integrated pulley part 473. Specifically, the washer 474a is provided adjacent to the Z1 side surface of the bearing part 472b of the bearing-integrated pulley part 472, which is placed in the Z1 side among the pully parts 472 and 473. The washer 474b is interposed in the axial direction (in the Z direction) between the Z2 side surface of the bearing part 472b of the bearing-integrated pulley part 472 and the Z1 side surface of the bearing part 473b of the bearing-integrated pulley part 473. The washer 474c is provided adjacent to the Z2 side surface of the bearing part 473b of the bearing-integrated pulley part 473, which is placed on the Z2 side among the pully part s 472 and 473.

As illustrated in FIG. 7, the bearing-integrated pulley 480 is provided corresponding to the driven member 441a. The bearing-integrated pulley 480 is provided in the in the shaft 42 side (the Y1 side) respective to the driven member 441a, in the direction that the shaft 42 extends (in the Y direction). The wire W1, which is led from the proximal end 42a of the shaft 42 toward the Y2 side, is wound around the bearing-integrated pulley 480 as being folded to a direction crossing the direction that the shaft 42 extends.

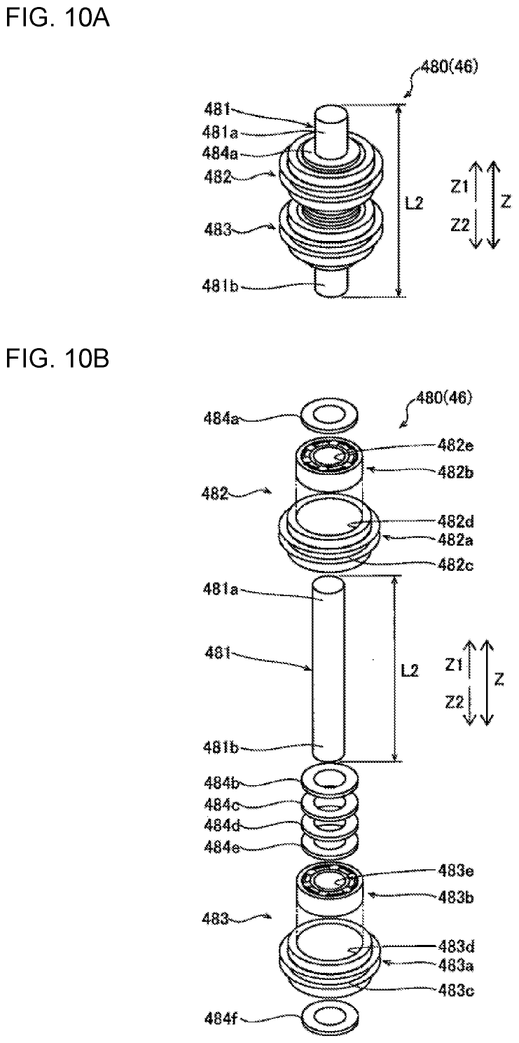

As illustrated in FIGS. 10A and 10B, the bearing-integrated pulley 480 includes the pulley shaft part 481, the plural (two) bearing-integrated pulley parts 482 and 483, and plural (six) washers 484a to 484f. The pulley shaft part 481 extends in the Z direction. The pulley shaft part 481 has the length L2 in the Z direction (the axial direction of the pulley shaft part). The length L2 is larger than the length L1 of the pulley shaft part 471 of the bearing-integrated pulley 470 in the Z direction, and substantially the same as the length L3 of the later described pulley shaft part 491 of the bearing-integrated pulley 490 in the Z direction. The bearing-integrated pulley part 482 is provided on (inserted through) the pulley shaft part 481. The bearing-integrated pulley part 483 is also provided on (inserted through) the pulley shaft part 481 such that with the bearing-integrated pulley parts 481 and 482 are provided side by side in the axial direction (the Z direction). The bearing-integrated pulley parts 482 and 483 are assembled to the pulley shaft part 481 in that order from the Z1 side toward the Z2 side. The first wire W1a and second wire W1b of the wire W1 are led from the shaft 42 through the bearing-integrated pulley part 482 and bearing-integrated pulley part 483 to the driven members 441a and are wound around and fixed to the driven members 441a.

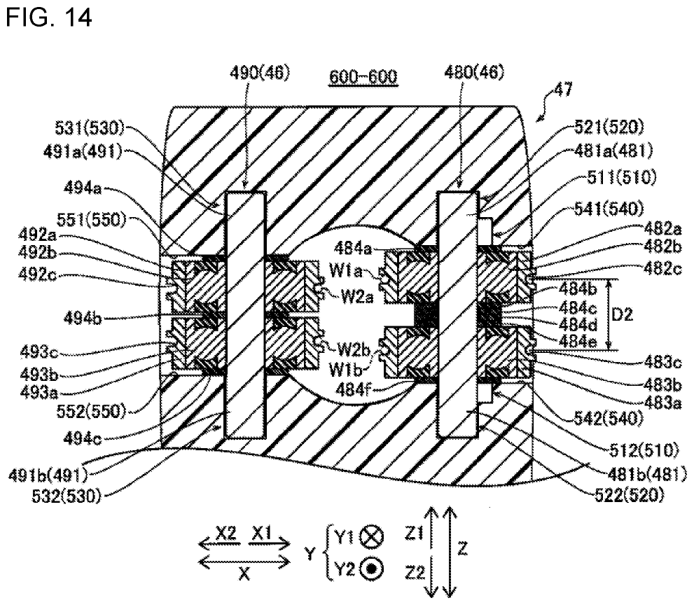

Specifically, the first wire W1a (see FIGS. 7 and 14), which is one side part of the wire W1, is wound around the bearing-integrated pulley part 482 to be folded in a substantially U-shape. The second wire W1b (see FIGS. 7 and 14), which is other side part of the wire W1, is wound around the bearing-integrated pulley part 483 to be folded in a substantially U-shape. The bearing-integrated pulley parts 482 and 483 are composed of same components (same members).

The bearing-integrated pulley part 482 (483) includes a pulley part 482a (483a) and a bearing part 482b (483b). The pulley part 482a includes a pulley groove 482c (483c) on its outer circumference. Around the pulley groove 482c (483c), the first wire W1a (second wire W1b) of the wire W1 is wound. The pulley part 482a (483a) includes a through-hole 482d (483d) penetrating in the Z direction and forming an inner circumference of the pulley part 482a (483a). The bearing part 482b (483b) is inserted in and fitted to the through-hole 482d (483d). The bearing part 482b (483b) is a ball bearing for example and its outer circumference is engaged with the through-hole 482d (483d) of the pulley part 482a (483a). The bearing part 482b (483b) holds the pulley part 482a (483a) rotatably about the rotational axis extending in the Z direction. The bearing part 482b (483b) includes a through-hole 482e (483e) which penetrates in the Z direction and which the pulley shaft part 481 is inserted in and fitted to.

The washers 484a and 484f are respectively provided on the Z1 side of the bearing-integrated pulley part 482 and on the Z2 side of the bearing-integrated pulley part 483. Specifically, the washer 484a is provided adjacent to the Z1 side surface of the bearing part 482b of the bearing-integrated pulley part 482, which is placed in the Z1 side among the pully parts 482 and 483. The washer 484f is provided adjacent to the Z2 side surface of the bearing part 483b of the bearing-integrated pulley part 483, which is placed in the Z2 side among the pully parts 482 and 483. The washers 484b to 484e are provided between the bearing-integrated pulley part 482 and the bearing-integrated pulley part 483. Specifically, the washers 484b to 484e are interposed in the axial direction (the Z direction) between the Z2 side surface of the bearing part 482b of the bearing-integrated pulley part 482 and the Z1 side surface of the bearing part 483b of the bearing-integrated pulley part 483.

As illustrated in FIG. 7, the bearing-integrated pulley 490 is provided corresponding to the driven member 441b. The bearing-integrated pulley 490 is provided on the shaft 42 side (the Y1 side) respective to the driven member 441b, in the direction that the shaft 42 extends (in the Y direction). Around the bearing-integrated pulley 490, the wire W2, which is led from the proximal end 42a of the shaft 42 toward the Y2 side, is wound as being folded to a direction crossing the direction that the shaft 42 extends.

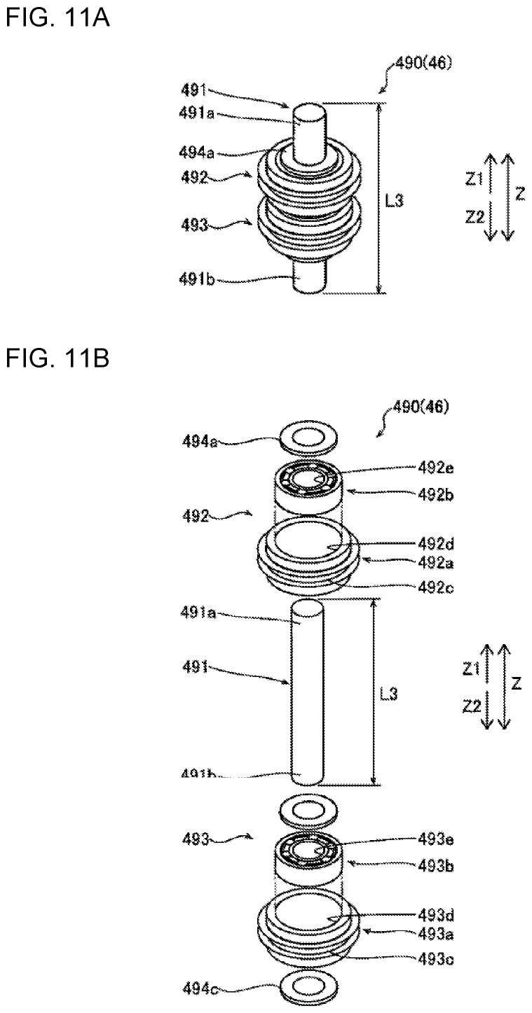

As illustrated in FIGS. 11A and 11B, the bearing-integrated pulley 490 includes the pulley shaft part 491, the plural (two) bearing-integrated pulley parts 492 and 493, and plural (three) washers 494a to 494c. The pulley shaft part 491 extends in the Z direction. The pulley shaft part 491 has the length L3 in the Z direction (the axial direction of the pulley shaft part). The length L3 is larger than the length L1 of the pulley shaft part 471 of the bearing-integrated pulley 470 in the Z direction and substantially the same as the length L2 of the pulley shaft part 481 of the bearing-integrated pulley 480 in the Z direction. The bearing-integrated pulley part 492 is provided on (inserted through) the pulley shaft part 491. The bearing-integrated pulley part 493 is also provided on (inserted through) the pulley shaft part 491 such that with the bearing-integrated pulley parts 491 and 492 are provided side by side in the axial direction (the Z direction). The bearing-integrated pulley parts 492 and 493 are assembled to the pulley shaft part 491 in that order from the Z1 side toward the Z2 side. The first wire W2a and second wire W2b of the wire W2 are led from the shaft 42 via the bearing-integrated pulley part 492 and bearing-integrated pulley part 493 to the driven member 441b and wound around and fixed to the driven member 441b.

Specifically, the first wire W2a (see FIGS. 7 and 14), which is one side part of the wire W2, is wound around the bearing-integrated pulley part 492 to be folded in a substantially U-shape. The second wire W2b (see FIGS. 7 and 14), which is the other side part of the wire W2, is wound around the bearing-integrated pulley part 493 to be folded in a substantially U-shape. The bearing-integrated pulley parts 492 and 493 are composed of same components (same members).

The bearing-integrated pulley part 492 (493) includes a pulley part 492a (493a) and a bearing part 492b (493b). The pulley part 492a includes a pulley groove 492c (493c) on its outer circumference. Around the pulley groove 492c (493c), the first wire W2a (second wire W2b) of the wire W2 is wound. The pulley part 492a (493a) includes a through-hole 492d (493d) penetrating in the Z direction and forming an inner circumference of the pulley part. The bearing part 492b (493b) is inserted in and fitted to the through-hole 492d (493d). The bearing part 492b (493b) is a ball bearing for example and its outer circumference is engaged with the through-hole 492d (493d) of the pulley part 492a (493a). The bearing part 492b (493b) holds the pulley part 492a (493a) rotatably about the rotational axis extending in the Z direction. The bearing part 492b (493b) includes a through-hole 492e (493e) which penetrates in the Z direction and which the pulley shaft part 491 is inserted in and fitted to.

The washers 494a to 494c are respectively provided on the Z1 side of the bearing-integrated pulley part 492, between the bearing-integrated pulley part 492 and bearing-integrated pulley part 493, and on the Z2 side of the bearing-integrated pulley part 493. Specifically, the washer 494a is provided adjacent to the Z1 side surface of the bearing part 492b of the bearing-integrated pulley part 492, which is placed in the Z1 side among the pulley parts 492 and 493. The washer 494b is interposed in the axial direction (in the Z direction) between the Z2 side surface of the bearing part 492b of the bearing-integrated pulley part 492 and the Z1 side surface of the bearing part 493b of the bearing-integrated pulley part 493. The washer 494c is provided adjacent to the Z2 side surface of the bearing part 493b of the bearing-integrated pulley part 493, placed in the Z2 side among the pulley parts 492 and 493.

According to an embodiment, the bearing-integrated pulleys 470, 480 and 490 have a configuration that at least a part of the members are common (are composed of same members). With this arrangement, types of the members composing the surgical instrument 40 can be reduced. Specifically, the bearing-integrated pulley parts 472, 482 and 492 in the bearing-integrated pulleys 470, 480 and 490 are composed of common members, which means the bearing-integrated pulley parts 472, 482 and 492 have the same size and shape. The bearing-integrated pulley parts 473, 483 and 493 in the bearing-integrated pulleys 470, 480 and 490 are also composed of common members, which means the bearing-integrated pulley parts 473, 483 and 493 have the same size and shape. With this configuration, since the bearing-integrated pulley part 472, 482, 492, 473, 483, 493, which are larger members of the members composing the bearing-integrated pulleys 470, 480 and 490, can be made common, types of the members composing the bearing-integrated pulleys 470, 480 and 490 can be effectively reduced. The washers 474a to 474c, 484a to 484f, and 494a to 494c in the bearing-integrated pulleys 470, 480 and 490 are also composed of common members, which means the washers 474a to 474c, 484a to 484f, and 494a to 494c have the same size and shape.

As illustrated in FIGS. 6, 7, and 12, the bearing-integrated pulley 470 and bearing-integrated pulley 480 are arranged side by side in the direction that the shaft 42 extends (in the Y direction). With this configuration, both of the bearing-integrated pulleys 470 and 480 can be arranged at positions where the wire W led from the shaft 42 can be easily wound. The bearing-integrated pulley 470 is positioned on the shaft 42 side (the Y1 side) respective to the bearing-integrated pulley 480, and the bearing-integrated pulley 480 is positioned in the side (the Y2 side) opposite to the shaft 42 side respective to the bearing-integrated pulley 470.

The bearing-integrated pulley 470 and bearing-integrated pulley 480 are positioned being displaced from each other in the direction (the X direction) substantially orthogonal to the direction that the shaft 42 extends, as seen in the direction (the Z direction) that the pulley shaft part 471 (481) extends. This configuration can prevent the wires W1 and W3 from interfering with each other since a path of the wire W3 to be wound around the bearing-integrated pulley 470 and a path of the wire W1 to be wound around the bearing-integrated pulley 480 can be displaced from each other in the X direction.

Specifically, the bearing-integrated pulley 470 positioned in the Y1 side is arranged being displaced in the X1 side (the side away from the shaft 42 in the X direction) respective to the bearing-integrated pulley 480. The bearing-integrated pulley 480 positioned in the Y2 side is arranged in the X2 side (the side closer to the shaft 42 in the X direction) respective to the bearing-integrated pulley 470. The bearing-integrated pulley 470 and bearing-integrated pulley 480 are arranged so that only a part of the respective pulley shaft parts 471, 481 overlaps as seen from the direction that the shaft 42 extends (the Y direction). This configuration can prevent the surgical instrument 40 from being enlarged in the X direction even in a case where the bearing-integrated pulleys 470 and 480 are arranged being shifted in the X direction since the X-direction shifting amount between the bearing-integrated pulley 470 and bearing-integrated pulley 480 can be made small.

The bearing-integrated pulley 480 and bearing-integrated pulley 490 are arranged side by side in a direction (the X direction) substantially orthogonal to the direction that the shaft 42 extends as seen in the direction (the Z direction) that the pulley shaft part 481 (491) extends. With this configuration, the bearing-integrated pulleys 480 and 490 can be arranged at positions where the wire W led from the shaft 42 can be easily wound. The bearing-integrated pulley 480 is arranged in the X1 side respective to the bearing-integrated pulley 490 and the bearing-integrated pulley 490 is arranged in the X2 side respective to the bearing-integrated pulley 480. The bearing-integrated pulleys 470, 480 and 490 are arranged in a substantially L shape as seen from the direction that the pulley shaft part 471 (481, 491) extends (the Z direction).

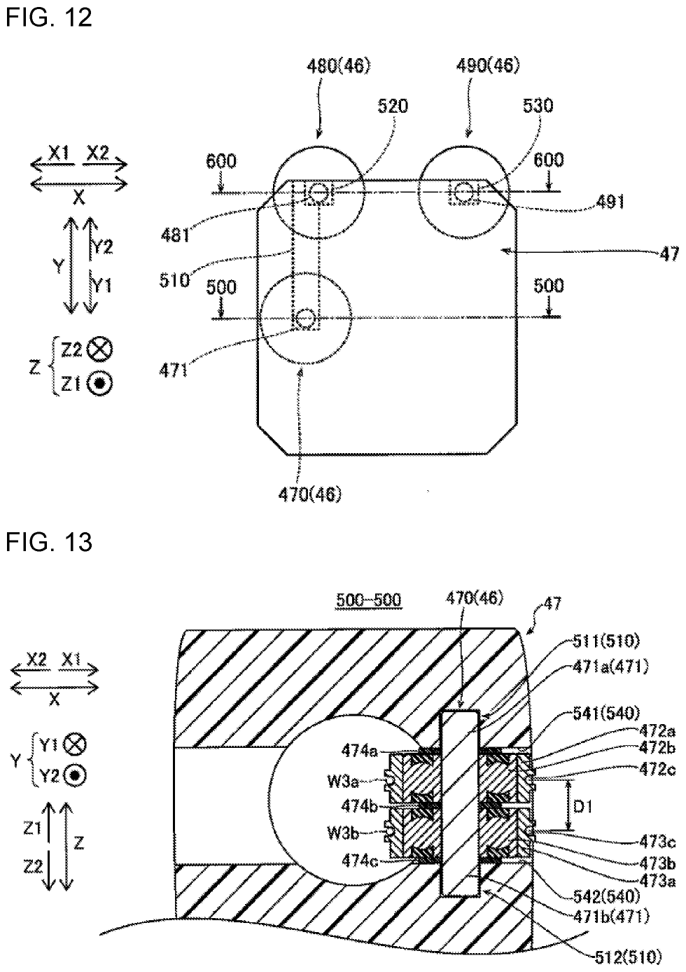

As illustrated in FIGS. 6, 7 and 12 to 14, the surgical instrument 40 includes a resin pulley retainer 47 that retains the bearing-integrated pulleys 470, 480 and 490. The pulley retainer 47 is provided on the base body 43 and is formed in a block shape or the like. The bearing-integrated pulleys 470, 480 and 490 are inserted to the pulley retainer 47 in a direction from the Y2 side to the Y1 side and held in the pulley retainer 47. Specifically, the pulley retainer 47 includes retaining grooves 510, 520 and 530 (see FIGS. 12, 13, and 14) that extend in the direction from the Y2 side to the Y1 side so that the bearing-integrated pulleys 470, 480 and 490 can be inserted from the Y2 side to the Y1 side.

As illustrated in FIGS. 12 and 13, the retaining groove 510 retains the pulley shaft part 471 of the bearing-integrated pulley 470, which is inserted from the Y2 side to the Y1 side. Specifically, the retaining groove 510 includes a first groove 511 and a second groove 512. The first groove 511 retains an end 471a in the Z1 side (one axial end 471a) of the pulley shaft part 471 of the bearing-integrated pulley 470 and the second groove 512 retains an end 471b in the Z2 side (the other axial end 471b) of the pulley shaft part 471 of the bearing-integrated pulley 470. The first groove 511 is formed being recessed in the Z1 side corresponding to the end 471a in the Z1 side of the pulley shaft part 471. The second groove 512 is formed being recessed in the Z2 side corresponding to the end 471b in the Z2 side in the pulley shaft part 471. The first groove 511 and second groove 512 are respectively formed extending in the Y direction from an insertion end in the Y2 side to a retaining position of the bearing-integrated pulley 470 in the Y1 side.

According to an embodiment, a position of the bearing-integrated pulley 470 retained in the retaining groove 510 is fixed in the retaining groove 510 by means of tension of the wire W3. With this configuration, since the bearing-integrated pulley 470 can be positioned using the tension of the wires W3, an increase of the number of parts to fix the position of the bearing-integrated pulley 470 can be prevented or suppressed. Specifically, the bearing-integrated pulley 470 is positioned in the retaining groove 510 by that the pulley shaft part 471 is pressed to a side face of the retaining groove 510 (a side face in the Y1 side and/or a side face in the X1 side of the retaining groove 510, for example) by the tension of the wire W3. The end in the Y1 side of the retaining groove 510 is positioned in the Y1 side respective to the retaining groove 520 so that the bearing-integrated pulley 470 is arranged in the Y1 side with respect to the bearing-integrated pulley 480.

As illustrated in FIGS. 12 and 14, the retaining groove 520 retains the pulley shaft part 481 of the bearing-integrated pulley 480 which is inserted from the Y2 side toward the Y1 side. Specifically, the retaining groove 520 includes the first and second grooves 521 and 522 that respectively retain the end 481a in the Z1 side (one axial end 481a) and the end 481b in the Z2 side (the other axial end 481b) of the pulley shaft part 481 of the bearing-integrated pulley 480. The first groove 521 is formed being recessed in the Z1 side corresponding to the end 481a in the Z1 side of the pulley shaft part 481. The second groove 522 is formed being recessed in the Z2 side corresponding to the end 481b in the Z2 side of the pulley shaft part 481. The first groove 521 and second groove 522 respectively positioned near an insertion end in the Y2 side.

The position of the bearing-integrated pulley 480 retained in the retaining groove 520 is fixed in the retaining groove 520 using tension of the wire W1. Specifically, the position of the bearing-integrated pulley 480 is fixed in the retaining groove 520 as the pulley shaft part 481 of the bearing-integrated pulley 480 is pressed to the side face of the retaining groove 520 (the side face in the Y1 side and/or the side face in the X1 side of the retaining groove 520 for example) using the tension of the wire W1.

The retaining groove 530 is arranged side by side with the retaining groove 520 in the X direction as seen from the Z direction. The retaining groove 530 retains the pulley shaft part 491 of the bearing-integrated pulley 490, which is inserted from the Y2 side toward the Y1 side. Specifically, the retaining groove 530 includes a first groove 531 and a second groove 532. The first groove 531 and second groove 532 respectively retain an end 491a in the Z1 side (one axial end 491a) and an end 491b in the Z2 side (the other axial end 491b) of the pulley shaft part 491 of the bearing-integrated pulley 490. The first groove 531 is formed being recessed in the Z1 side corresponding to the end 491a in the Z1 side of the pulley shaft part 491. The second groove 532 is formed being recessed in the Z2 side corresponding to the end 491b in the Z2 side of the pulley shaft part 491. The first and second grooves 531 and 532 are respectively positioned near an insertion end in the Y2 side.

The position of the bearing-integrated pulley 490 retained by the retaining groove 530 is fixed in the retaining groove 530 by tension of the wire W2. Specifically, the position of the bearing-integrated pulley 490 is fixed in the retaining groove 530 as the pulley shaft part 491 is pressed to the side face of the retaining groove 530 (the side face in the Y1 side and/or the side face in the X2 side of the retaining groove 530 for example) by tension of the wire W2.

As illustrated in FIGS. 13 and 14, the pulley retainer 47 includes removed portions 540 and 550 or cutouts. The removed portions 540 and 550 are formed by cutting the pulley retainer 47 from the Y2 side to the Y1 side so that the bearing-integrated pulleys 470, 480 and 490 can be inserted from the Y2 side to the Y1 side in the removed portions. The removed portion 540 is provided corresponding to the bearing-integrated pulleys 470 and 480. Specifically, the removed portion 540 is provided so that the bearing-integrated pulley parts 472 and 473 and the washers 474a to 474c of the bearing-integrated pulley 470 can be inserted in the removed portion 540. The removed portion 540 is formed so that its end surface 541 in the Z1 side of the removed portion 540 is in contact with the Z1 side surface of the washer 474a of the bearing-integrated pulley 470. The removed portion 540 is formed so that its end surface 542 in the Z2 side of the removed portion 540 is in contact with the Z2 side surface of the washer 474c of the bearing-integrated pulley 470. With this configuration, the removed portion 540 positions (fixes) the bearing-integrated pulley 470 in the Z direction.

In a similar manner, the removed portion 540 is formed so that bearing-integrated pulley parts 482 and 483 and the washers 484a to 484f of the bearing-integrated pulley 480 can be inserted. The removed portion 540 is formed so that its end face 541 in the Z1 side of the removed portion 540 is in contact with the Z1 side surface of the washer 484a of the bearing-integrated pulley 480. The removed portion 540 is formed so that its end surface 542 in the Z2 side of the removed portion 540 is in contact with the Z2 side surface of the washer 484f of the bearing-integrated pulley 480. With this configuration, the removed portion 540 positions (fixes) the bearing-integrated pulley 480 in the Z direction.

The removed portion 550 is provided corresponding to the bearing-integrated pulley 490. Specifically, the removed portion 550 is formed so that the bearing-integrated pulley parts 492 and 493 and the washers 494a to 494c of the bearing-integrated pulley 490 can be inserted. The removed portion 550 is formed so that its end surface 551 in the Z1 side of the removed portion 550 is in contact with the Z1 side surface of the washer 494a of the bearing-integrated pulley 490. The removed portion 550 is also formed so that its end surface 552 in the Z2 side of the removed portion 550 is in contact with the Z2 side surface of the washer 494c of the bearing-integrated pulley 490. With this configuration, the removed portion 550 positions (fixes) the bearing-integrated pulley 490 in the Z direction.

According to an embodiment, an interval or a distance in the Z direction between the bearing-integrated pulley parts 472 and 473 of the bearing-integrated pulley 470 is different from an interval or a distance in the Z direction between the bearing-integrated pulley parts 482 and 483 in bearing-integrated pulley 480. This configuration prevents the wires W1 and W3 from interfering each other since the path that the wire W3 is wound around the bearing-integrated pulley 470 and the path that the wire W1 is wound around the bearing-integrated pulley 480 can be displaced from each other in the Z direction. Specifically, the distance D1 in the Z direction between the bearing-integrated pulley part 472 and bearing-integrated pulley part 473 of the bearing-integrated pulley 470 arranged in the Y1 direction is smaller than the distance D2 in the Z direction between the bearing-integrated pulley part 482 and bearing-integrated pulley part 483 of the bearing-integrated pulley 480 arranged in the Y2 side. The distance D1 (D2) is a length in the Z-direction from the pulley groove 472c (482c) of the bearing-integrated pulley part 472 (482) to the pulley groove 473c (483c) of the bearing-integrated pulley part 473 (483).

The distance in the Z-direction between the bearing-integrated pulley part (472, 482) and bearing-integrated pulley part (473, 483) is adjusted with the number of the washers (474b, 484b to 484e) between the bearing-integrated pulley part (472, 482) and bearing-integrated pulley part (473, 483). Therefore, as described above, the number of washers (474b) between the bearing-integrated pulley part 472 and bearing-integrated pulley part 473 of the bearing-integrated pulley 470 is smaller than the number of the washers (484b to 484e) between the bearing-integrated pulley part 482 and bearing-integrated pulley part 483 of the bearing-integrated pulley 480. In the bearing-integrated pulley 480, the number of washers (484b to 484e) between the bearing-integrated pulley part 482 and bearing-integrated pulley part 483 of the bearing-integrated pulley 480 is greater than the number of washers (474b) between the bearing-integrated pulley part 472 and bearing-integrated pulley part 473.

In addition, according to an embodiment, the lengths of the pulley shaft parts (471, 481) of the bearing-integrated pulley 470 and bearing-integrated pulley 480 are different from each other. This configuration prevents the bearing-integrated pulley 470 and bearing-integrated pulley 480 from being mistakenly assembled during an assemble operation since the bearing-integrated pulley 470 and bearing-integrated pulley 480, which are arranged side by side in the Y direction, can be easily distinguished. Specifically, the length of the pulley shaft part (471) of the bearing-integrated pulley 470 is smaller than the pulley shaft part (481) of the bearing-integrated pulley 480. With this configuration, since the bearing-integrated pulley 470 provided in the Y1 side can be made smaller, the operation to assemble the bearing-integrated pulley 470 in the Y1 side can be made easier.

### [Modification]

It should be understood that the one or more embodiments described above are illustrated by way of example in every respect and not limit the invention. The scope of the invention is indicated by claims, not by explanation of the one or more embodiments described above, and includes equivalents to the claims and all alterations (modification) within the same.

For example, In the one or more embodiments described above, three bearing-integrated pulleys are arranged in the surgical instrument; however, the invention is not limited to this. For example, in an embodiment or a modification, only one bearing-integrated pulley may be provided in the surgical instrument or plural (other than three) bearing-integrated pulleys may be provided. In addition, all pulleys provided to the surgical instrument do not need to be bearing-integrated pulleys and only one or more pulley in the all pulleys that has a greater load may be provided as a bearing-integrated pulley.

In the one or more embodiments described above, the number of the bearing-integrated pulley corresponds to the number of plural driven members around which the wires (elongated element) are wound and fixed; however, the invention is not limited to this. In an embodiment or a modification, the number of the bearing-integrated pulley does not always need to correspond to the number of plural driven members around which the elongated elements are wound and fixed. The number of the bearing-integrated pulleys may be fewer than the corresponding number of driven members around which the elongated elements are wound and fixed.

In the one or more embodiments described above, the distance between the bearing-integrated pulley parts (the first bearing-integrated pulley part and second bearing-integrated pulley part) differs in the bearing-integrated pulleys arranged side by side in the Y direction (the first bearing-integrated pulley and second bearing-integrated pulley); however, the invention is not limited to this. For example, in an embodiment or a modification, the distance between the first bearing-integrated pulley part and second bearing-integrated pulley part may be the same in the first bearing-integrated pulley and second bearing-integrated pulley.

Further, In the one or more embodiments described above, the length (axial length) of the pulley shaft part differs in the bearing-integrated pulleys arranged side by side in the Y direction (the first bearing-integrated pulley and second bearing-integrated pulley); however, the invention is not limited to this. For example, in an embodiment or a modification, the length (axial length) of the pulley shaft part may be the same in the first bearing-integrated pulley and second bearing-integrated pulley.

In the one or more embodiments described above, the length of the pulley shaft part is smaller in the bearing-integrated pulley (first bearing-integrated pulley) in the Y1 side than that in the bearing-integrated pulley (second bearing-integrated pulley) in the Y2 side; however, the invention is not limited to this. For example, in an embodiment or a modification, the length of the pulley shaft part may be smaller in the second bearing-integrated pulley than the first bearing-integrated pulley.

In the one or more embodiments described above, the bearing-integrated pulley in the Y1 side (first bearing-integrated pulley) and the bearing-integrated pulley in the Y2 side (second bearing-integrated pulley) are displaced from each other in the X direction; however, the invention is not limited to this. For example, in an embodiment or a modification, the first bearing-integrated pulley and second bearing-integrated pulley does not always need to be displaced from each other in the X direction.

In the one or more embodiments described above, at least a part of the plural bearing-integrated pulleys is composed of a common member; however, the invention is not limited to this. For example, in an embodiment or a modification, the at lease a part of the bearing-integrated pulleys does not need to be composed of a common member.

In the one or more embodiments described above, the bearing-integrated pulley is provided whose position is fixed by tension of the wire (elongated element); however, the invention is not limited to this. For example, in an embodiment or a modification, the bearing-integrated pulley may be fixed without using tension of the elongated element.

In the one or more embodiments described above, the adaptor and drape are provided independently of each other, but the invention is not limited thereto. For example, in an embodiment or a modification, the adaptor and drape may be provided integrally. That is, the adaptor may be an adaptor integrated with the drape.

1. A surgical instrument to be detachably connected to a robot arm of a robotic surgical system, the surgical instrument comprising:
a base body;
a treatment tool;
an elongated shaft whose proximal end is attached to the base body and whose distal end is provided with the treatment tool;
a driven member provided in the base body to be rotatable about a rotational axis with respect to the base body;
a bearing-integrated pulley; and
an elongated element extending through the shaft to operate the treatment tool, wherein the elongated element is led from the shaft to the driven member through the bearing-integrated pulley and wound around and fixed to the driven member.

2. The surgical instrument according to claim 1, wherein
the bearing-integrated pulley includes a pulley shaft part, and first and second bearing-integrated pulley parts attached to the pulley shaft part, and
one side of the elongated element is led from the shaft through the first bearing-integrated pulley part to the driven member and wound around the driven member and the other side of the elongated element is led from the shaft through the second bearing-integrated pulley part to the driven member and wound around and fixed to the driven member.

3. The surgical instrument according to claim 2, wherein
the one side of the elongated element is wound around the first bearing-integrated pulley part to be folded in a U-shape at the first bearing-integrated pulley part and the other side of the elongated element is wound around the second bearing-integrated pulley part to be folded in a U-shape at the second bearing-integrated pulley part.

4. The surgical instrument according to claim 2, further comprising
a pulley retainer that is provided at the base body to retain the bearing-integrated pulley,
wherein the pulley retainer includes a retaining groove that retains the pulley shaft part of the bearing-integrated pulley.

5. The surgical instrument according to claim 2, wherein
the bearing-integrated pulley comprises a first bearing-integrated pulley and a second bearing-integrated pulley that are arranged side by side in a direction that the shaft extends.

6. The surgical instrument according to claim 5, wherein
each of the first bearing-integrated pulley and the second bearing-integrated pulley includes the first bearing-integrated pulley part and the second bearing-integrated pulley part, and
a distance between the first bearing-integrated pulley part and the second bearing-integrated pulley part in the first bearing-integrated pulley is different from a distance between the first bearing-integrated pulley part and the second bearing-integrated pulley part in the second bearing-integrated pulley.

7. The surgical instrument according to claim 6, wherein
washers are provided between the first bearing-integrated pulley part and the second bearing-integrated pulley part in each of the first bearing-integrated pulley and the second bearing-integrated pulley, and
a number of the washers provided between the first bearing-integrated pulley part and the second bearing-integrated pulley part in the first bearing-integrated pulley is different from a number of the washers provided between the first bearing-integrated pulley part and the second bearing-integrated pulley part in the second bearing-integrated pulley.

8. The surgical instrument according to claim 5, wherein
a length of the pulley shaft part in the first bearing-integrated pulley is different from a length of the pulley shaft part in the second bearing-integrated pulley.

9. The surgical instrument according to claim 8, wherein
the first bearing-integrated pulley is positioned closer to the shaft than the second bearing-integrated pulley is, and
the length of the pulley shaft part in the first bearing-integrated pulley is shorter than the length of the pulley shaft part in the second bearing-integrated pulley.

10. The surgical instrument according to claim 5, wherein
the first bearing-integrated pulley and the second bearing-integrated pulley are decentered from each other in a direction orthogonal to the direction that the shaft extends, as seen in the direction that the pulley shaft part extends.

11. The surgical instrument according to claim 10, wherein
the first bearing-integrated pulley and the second bearing-integrated pulley are arranged such that only a part of the pulley shaft part of the first bearing-integrated pulley and the pulley shaft part of the second bearing-integrated pulley overlaps as seen in the direction that the shaft extends.

12. The surgical instrument according to claim 5, wherein
at least a part of members of the first bearing-integrated pulley and the second bearing-integrated pulley have a same size and shape.

13. The surgical instrument according to claim 12, wherein
the first bearing-integrated pulley part and the second bearing-integrated pulley part in the first bearing-integrated pulley and the second bearing-integrated pulley have a same size and shape.

14. The surgical instrument according to claim 5, wherein
the first bearing-integrated pulley is positioned closer to the shaft than the second bearing-integrated pulley is, and
around the first bearing-integrated pulley, the elongated element led from the proximal end of the shaft is wound to be fold back toward the shaft.

15. The surgical instrument according to claim 5, wherein
the driven member includes a first driven member, a second driven member, and a third driven member, and
the bearing-integrated pulley includes the first bearing-integrated pulley, the second bearing-integrated pulley, and a third bearing-integrated pulley corresponding to the first driven member, the second driven member, and the third driven member, respectively.

16. The surgical instrument according to claim 15, wherein
the second bearing-integrated pulley is positioned further away from the shaft than the first bearing-integrated pulley is, and
the third bearing-integrated pulley is arranged side by side with the second bearing-integrated pulley in a direction orthogonal to the direction that the shaft extends as seen in the direction that the pulley shaft part extends.

17. The surgical instrument according to claim 15, wherein
the treatment tool includes a pair of jaws and a wrist portion,
the elongated element includes a first wire whose end portions are wound around and fixed to the first driven member, a second wire whose end portions are wound around and fixed to the second driven member, and a third wire whose end portions are wound around and fixed to the third driven member, and
the first wire is operated by the first driven member to rotate the wrist portion, and the second and third wires are operated by the second and third driven members respectively to open and close the pair of jaws.

18. The surgical instrument according to claim 1, wherein
a position of the bearing-integrated pulley is fixed using tension of the elongated element.

19. A robotic surgical system, comprising:
a robot arm; and
a surgical instrument detachably connected to the robot arm, wherein
the surgical instrument includes
a base body,
a treatment tool,
an elongated shaft whose proximal end is attached to the base body and whose distal end is provided with the treatment tool,
a driven member provided at the base body to be rotatable about a rotational axis with respect to the base body,
a bearing-integrated pulley, and
an elongated element extending through the shaft to operate the treatment tool, wherein the elongated element led from the shaft to the driven member through the bearing-integrated pulley and wound around and fixed to the driven member.

20. A method of fixing a bearing-integrated pulley around which a wire to operate a treatment tool is wound, the treatment tool provided at a distal end of a shaft of a surgical instrument to be detachably connected to a robot arm, the method comprising:
retaining a pulley shaft part of the bearing-integrated pulley by inserting the pulley shaft part of the bearing-integrated pulley into a retaining groove formed on a pulley retainer, and
pressing the pulley shaft part onto a side surface of the retaining groove using tension of the wire.
